(19) United States
(12) Patent Application Publication
(10) Pub. No.: US 2026/0321432 A1
(43) Pub. Date: Oct. 15, 2026

(54) SYSTEMS AND METHODS FOR REPRODUCTIVE PATHWAY FORECASTING AND COORDINATION

(71) Applicants: Rachel Beth KULE, Boca Raton, FL (US); Matthew Dane KULE, Boca Raton, FL (US)

(72) Inventors: Rachel Beth KULE, Boca Raton, FL (US); Matthew Dane KULE, Boca Raton, FL (US)

(21) Appl. No.: 19/643,038

(22) Filed: Apr. 9, 2026

Related U.S. Application Data
(60) Provisional application No. 63/786,439, filed on Apr. 10, 2025.

(51) Int. Cl. G16H 50/30; G16H 10/40; G16H 10/60; G16H 50/70; G16H 80/00

(52) U.S. Cl. CPC G16H 50/30; G16H 10/40; G16H 10/60; G16H 50/70; G16H 80/00

(57) ABSTRACT

In accordance with the present disclosure, a system for reproductive pathway forecasting and coordination includes a processor and a memory storing instructions that, when executed by the processor, cause the system to receive patient-specific fertility data from a user device including known indicators and biomarkers of fertility, analyze the patient-specific fertility data, generate a predictive fertility forecast assessment including an estimated in-vitro fertilization success rate, an associated treatment cost, and a projected treatment timeline, present the predictive fertility forecast and at least one alternative fertility treatment option including surrogacy on a user interface.

### CROSS-REFERENCE TO RELATED APPLICATION

This application claims the benefit of, and priority to, U.S. Provisional Patent Application Ser. No. 63/786,439 filed on Apr. 10, 2025, the entire content of which is hereby incorporated by reference.

### TECHNICAL FIELD

The present invention relates generally to systems and methods for fertility treatment management and, more specifically, to platforms and techniques for predictive forecasting of in-vitro fertilization (IVF) outcomes, fertility treatment planning, and surrogate matching, using artificial intelligence (AI) and patient-specific data.

### BACKGROUND

Assisted reproductive technologies (ART), including in-vitro fertilization (IVF), have become widely used in addressing infertility. IVF is a medical procedure in which an egg is fertilized by sperm outside the body and then implanted into the uterus. According to the U.S. Department of Health and Human Services, IVF accounts for more than 99% of all ART procedures. Despite its prevalence, the IVF process remains time-consuming, expensive, and emotionally demanding. Patients face a wide range of medical, logistical, and financial challenges, often without sufficient guidance, including regarding grief and coping, or individualized predictive tools. Success rates for IVF vary significantly depending on patient-specific factors such as age, biomarkers, uterine health, partner or donor characteristics, and medical provider protocols. For instance, while some patients may have a 40-60% chance of success, others, especially those over 40 years of age, face rates as low as 7%. Additionally, IVF cycles can cost tens of thousands of dollars, frequently requiring multiple attempts before achieving pregnancy, if at all.

In some cases, alternative pathways such as egg or sperm donation and gestational surrogacy may be pursued. Surrogacy refers to an arrangement where a woman carries and delivers a child for another individual or couple, often using an embryo created via IVF. However, these options often introduce further complexity, financial burden, and emotional considerations. Surrogacy, while statistically more successful in terms of live birth outcomes, typically involves multi-year wait times and high agency fees, limiting accessibility for many intended parents.

Compounding these challenges is the lack of consistent, integrated communication between patients and medical providers. Errors in timing, medication, or recordkeeping can result in cycle cancellation or failed embryo transfer, leading to avoidable losses in time, money, and reproductive opportunity. Providers face challenges managing increasingly complex patient cases, high communication demands, and pressure to deliver successful outcomes under time-sensitive conditions. Inconsistent patient recordkeeping, incomplete patient understanding, and decentralized data sources hinder efficient treatment planning and continuity of care. Fertility clinics must manually track individualized patient timelines, medication protocols, diagnostic results, and evolving options, creating operational inefficiencies and increasing the risk of human error.

Accordingly, there remains a need for a system and method that can provide patients with individualized forecasts for IVF success, cost, and duration; reduce avoidable treatment delays and medical errors through structured, real-time communication; and optimize matching with surrogates or donors when needed. Such a system should support both patients and providers throughout the fertility journey using intelligent, adaptive technology, while also enabling providers to streamline communication, enhance coordination across care teams, and improve clinical outcomes by leveraging patient-specific data in a unified interface.

### SUMMARY

In accordance with the present disclosure, a system for reproductive pathway forecasting and coordination includes a processor and a memory storing instructions that, when executed by the processor, cause the system to receive patient-specific fertility data from a user device including known indicators and biomarkers of fertility, analyze the patient-specific fertility data, generate a predictive fertility forecast assessment including an estimated in-vitro fertilization success rate, an associated treatment cost, and a projected treatment timeline, present the predictive fertility forecast and at least one alternative fertility treatment option including surrogacy on a user interface.

In an aspect, the known indicators and biomarkers of fertility may include anti-mullerian hormone levels, follicle-stimulating hormone levels, age, weight, height, advanced genetic embryo testing results, semen analysis data, or combinations thereof.

In an aspect, the system may include instructions that may generate an updated predictive fertility forecast based on recruitment of healthy eggs, fertilization of the healthy eggs to create an embryo, or the success of embryo development in a laboratory.

In an aspect, the system may include instructions that may facilitate interactive communication between the user and at least one medical provider regarding the predictive fertility forecast and the alternative fertility treatment option.

In an aspect, the system may include instructions that may receive a request to engage in surrogacy from an intended parent profile, receive surrogate candidate data from a plurality of potential surrogates, evaluate the surrogate candidate data against the intended parent profile to identify at least one surrogate match, display the surrogate match on the user interface, and enable a mutual match.

In an aspect, a mutual match may include approval of the surrogate match by the intended parent profile and approval of the intended parent profile by the surrogate match.

In an aspect, the interactive communication may include a shared framework between the user and the medical provider.

In an aspect, the shared framework may include direct messaging between a sender and a receiver.

In an aspect, the sender may categorize a direct message by topic and by a level of urgency for response.

In accordance with the present disclosure, a computer-implemented method for providing an in-vitro fertilization forecast includes receiving patient-specific fertility data from a user device including known indicators and biomarkers of fertility, analyzing the patient-specific fertility data, generating a predictive fertility forecast assessment including an estimated in-vitro fertilization success rate, an associated treatment cost, and a projected treatment timeline, presenting the predictive fertility forecast and at least one alternative fertility treatment option including surrogacy on a user interface.

In an aspect, the known indicators and biomarkers of fertility may include anti-mullerian hormone levels, follicle-stimulating hormone levels, age, weight, height, advanced genetic embryo testing results, semen analysis data, or combinations thereof.

In an aspect, the method may include generating an updated predictive fertility forecast based on recruitment of healthy eggs, fertilization of the healthy eggs to create an embryo, or success of embryo development in a laboratory.

In an aspect, the method may include facilitating interactive communication between the user and at least one medical provider regarding the predictive fertility forecast and the alternative fertility treatment option.

In an aspect, the method may include receiving a request to engage in surrogacy from an intended parent profile, receiving surrogate candidate data from a plurality of potential surrogates, evaluating the surrogate candidate data against the intended parent profile to identify at least one surrogate match, displaying the surrogate match on the user interface, and enabling a mutual match.

In an aspect, a mutual match may include approval of the surrogate match by the intended parent profile and approval of the intended parent profile by the surrogate match.

In an aspect, the interactive communication may include a shared framework between the user and the medical provider.

In an aspect, the shared framework may include direct messaging between a sender and a receiver.

In an aspect, the sender may categorize a direct message by topic and by a level of urgency for response.

### BRIEF DESCRIPTION OF THE DRAWINGS

A better understanding of the features and advantages of the present disclosure will be obtained by reference to the following detailed description that sets forth illustrative embodiments, in which the principles of the present disclosure are utilized, and the accompanying drawings of which:

FIG. 1 is a diagram of a system for reproductive pathway forecasting and coordination, in accordance with aspects of the present disclosure;

FIG. 2 is a block diagram of a controller for the system of FIG. 1, in accordance with aspects of the present disclosure;

FIG. 3 is a flow diagram illustrating a method for generating and updating individualized fertility forecasts using the system of FIG. 1, in accordance with aspects of the present disclosure;

FIG. 4 is a flow diagram illustrating a method for coordinating and matching surrogates of FIG. 3, using the system of FIG. 1, in accordance with aspects of the present disclosure; and

FIG. 5 is a flow diagram illustrating a method for facilitating communication between a user and at least one medical provider of FIG. 3, using the system of FIG. 1, in accordance with aspects of the present disclosure.

### DETAILED DESCRIPTION

The present disclosure relates generally to the system and method for predictive forecasting of in-vitro fertilization (IVF) outcomes, fertility treatment planning, and surrogate matching, using artificial intelligence (AI) and patient-specific data.

Although the present disclosure will be described in terms of specific examples, it will be readily apparent to those skilled in this art that various modifications, rearrangements, and substitutions may be made without departing from the spirit of the present disclosure.

For the purpose of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

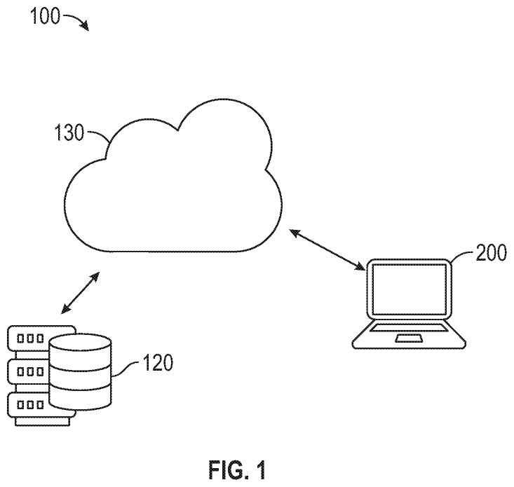

Referring to FIG. 1, an exemplary system 100 for reproductive pathway forecasting and coordination, includes a controller 200. The disclosed system 100 provides a technical solution to the problem of fragmented fertility care, unpredictable treatment outcomes, delays in communication between patients and providers, and the inefficiencies associated with surrogacy matching. The system 100 provides the benefit of delivering individualized forecasts and treatment support that improve clinical decision-making, reduce costly errors, and streamline the fertility process for both patients and providers. Additionally, it facilitates direct surrogate matching based on personalized criteria, thereby accelerating timelines, reducing financial burdens, and enhancing transparency for intended parents and surrogates alike.

It is contemplated that the controller 200 may run programs locally on the controller 200, remotely via a network 130, over the cloud, and/or on a remote server 120. The controller 200 may include but is not limited to, a mobile phone, a personal computer, a tablet, and/or other handheld devices. The disclosed system is configured to reduce the burden on fertility clinics and streamline treatment planning by providing intelligent fertility forecasting and coordination tools. Such tools enable a user to access individualized reproductive guidance, communicate with fertility providers, and manage surrogacy-related workflows in a centralized platform.

The network 130 may be wired or wireless and can utilize technologies such as Wi-Fi®, Ethernet, Internet Protocol, 3G, 4G, 5G, TDMA, CDMA, or other communication technologies. The network 130 may include, for example, but is not limited to, a cellular network, residential broadband, satellite communications, private network, the Internet, local area network, wide area network, storage area network, campus area network, personal area network, or metropolitan area network.

The term “application” may include a computer program and/or machine-readable instructions designed to perform particular functions, tasks, or activities for the benefit of a user. Application may refer to, for example, software running locally or remotely, as a standalone program or in a web browser, or other software that would be understood by one skilled in the art to be an application. An application may run on the controller 200, a server, or on a user device 140, including, for example, on a user device 140 or a client computer system 110. The configuration of FIG. 1 is exemplary, and variations are contemplated to be within the scope of the present disclosure.

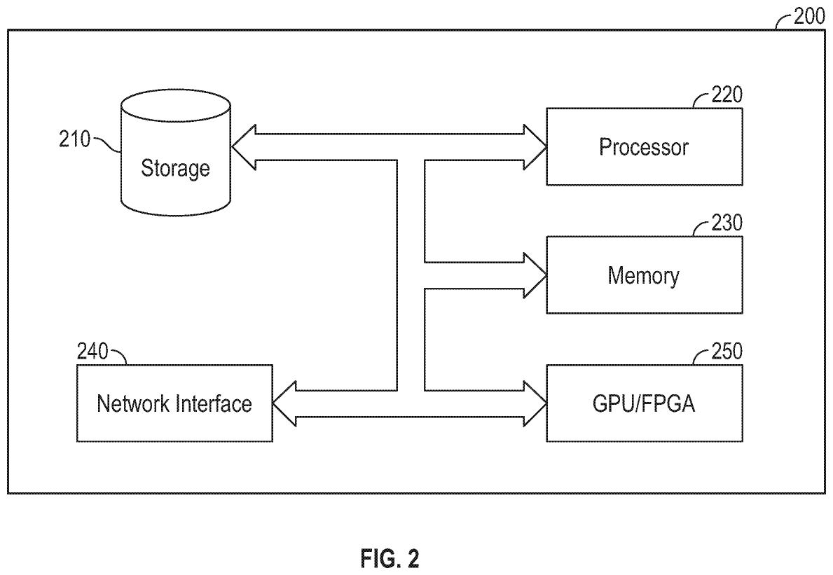

Referring now to FIG. 2, exemplary components in the controller 200 in accordance with aspects of the present disclosure include, for example, a database 210, one or more processors 220, at least one memory 230, and a network interface 240. In aspects, the controller 200 may include a graphical processing unit (GPU) 250, which may be used for processing machine learning models.

The database 210 can be located in storage. The term “storage” may refer to any device or material from which information may be capable of being accessed, reproduced, and/or held in an electromagnetic or optical form for access by a computer processor. Storage may be, for example, volatile memory such as RAM, non-volatile memory, which permanently holds digital data until purposely erased, such as flash memory, magnetic devices such as hard disk drives, and optical media such as a CD, DVD, Blu-ray disc, or the like. In various embodiments, data may be stored on the controller 200, including, for example, user preferences, historical data, and/or other data. The data can be stored in database 210 and sent via the system bus to the processor 220.

As will be described in more detail later herein, the processor 220 executes various processes based on instructions that can be stored in the server memory 230 and utilizing the data from the database 210. The illustration of FIG. 2 is exemplary, and it will be understood by persons skilled in the art that other components may exist in a controller 200. Such other components are not illustrated in FIG. 2 for clarity of illustration.

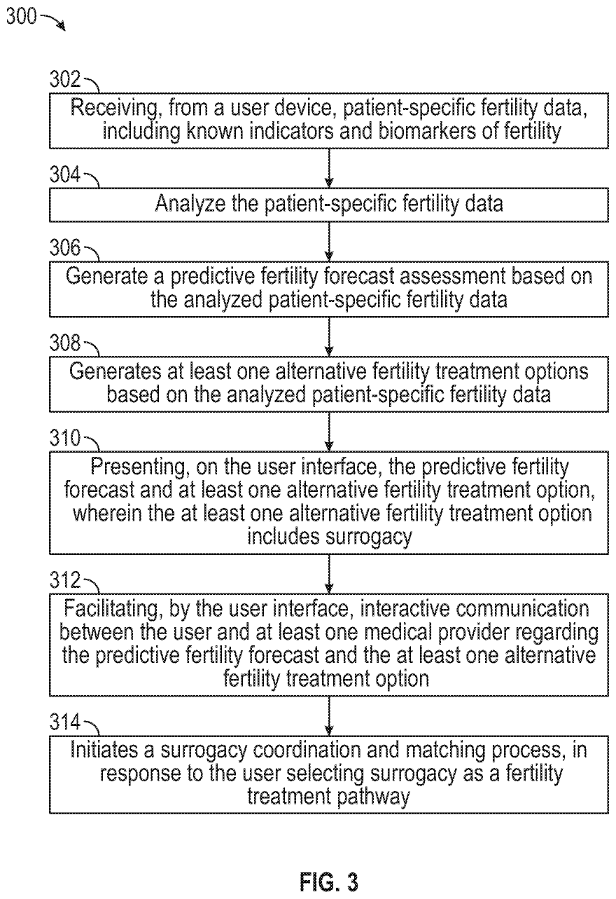

Referring to FIG. 3, a computer-implemented method 300 is illustrated for generating and updating individualized fertility forecasts using controller 200. The method 400 is executed by at least one processor 220 in communication with memory 230 storing instructions that, when executed, cause the controller 200 to perform the operations described herein. The disclosed method provides a technical solution to the technical problem of fragmented, manual, and non-predictive fertility planning by enabling a computing system to automatically ingest heterogeneous fertility data, apply artificial intelligence (AI)-based analysis, and generate adaptive, individualized fertility forecasts related to in-vitro fertilization (IVF) outcomes, treatment costs, and timelines. By integrating computing hardware, predictive modeling, and structured patient data, the invention improves the technological process by which reproductive treatment assessments are calculated, communicated, and updated, thereby reducing human error, improving continuity of care, and providing patients with a clearer, more consistent understanding of available fertility pathways, including the expected financial, emotional, and time commitments associated with each pathway, so that patients and providers can make more informed decisions and develop a more strategic treatment plan from the outset. In some embodiments, the AI-driven analysis includes feature extraction, weighting, and probabilistic modeling executed by trained machine-learning models stored in memory 230 and executed by processor 220.

At step 302, the controller 200 receives, from a user device 140, patient-specific fertility data including known indicators and biomarkers of fertility. The user device 140 may include a smartphone, tablet, or personal computer executing a patient-facing application. The patient-specific fertility data received at step 302 includes demographic information relating to the patient, such as age, height, weight, and body mass index, as well as reproductive history information including prior pregnancies, miscarriages, or live births. The received data further includes information identifying a treating physician or clinic, clinic location information, and publicly available physician or clinic success rate data, along with information indicating partner involvement or donor participation.

In addition to the demographic and historical information, the controller 200 requires receipt of diagnostic laboratory testing results corresponding to core fertility biomarkers. These required diagnostic inputs include Anti-Müllerian Hormone (AMH) levels and Follicle-Stimulating Hormone (FSH) levels obtained through laboratory blood testing. In aspects, the controller 200 requires receipt of ultrasound results detailing follicle amount and images of the uterine cavity, as well as MRI testing results. The diagnostic laboratory testing results may be uploaded through the user interface by the patient, manually entered or inputted by the patient, transmitted by a medical provider, or received through electronic integration with a laboratory or healthcare information system. The data is transmitted over network 130 and stored within database 210. Upon receipt of the patient-specific fertility data and the required biomarker testing results, the processor 220 initiates data normalization and categorization operations, including standardizing measurement units, organizing patient data into defined ranges, and applying weighting to fertility-related risk factors, thereby preparing the data for further computational analysis.

At step 304, the processor 220 analyzes the patient-specific fertility data by incorporating diagnostic and clinical biomarker information, received at step 302 by mining, aggregating, and evaluating the demographic information, laboratory biomarker results, diagnostic testing data, and medical records associated with the patient. This analysis is executed by processor 220, and in some embodiments is accelerated by GPU 250, using trained machine-learning models stored in memory 230. The AI-driven analysis integrates known fertility biomarkers and diagnostic testing results such that multiple data inputs converge to form a structured analytical representation of the patient's reproductive profile, which is used to assess reproductive potential and treatment feasibility.

The analysis includes evaluation of ovarian reserve and egg quality indicators based on Anti-Müllerian Hormone (AMH) levels, Follicle-Stimulating Hormone (FSH) levels, and patient age. The trained AI models are configured to recognize clinically established benchmarks, including that lower AMH levels in combination with elevated FSH levels and advanced age are indicative of diminished ovarian reserve and reduced egg quality, while higher AMH levels are generally associated with a greater ovarian reserve and increased likelihood of successful fertilization. The AI models further account for the biological role of FSH as a pituitary hormone that stimulates follicular growth and maturation, and analyze FSH values in relation to ovarian response expectations derived from historical outcome data. In addition to these biomarkers, the AI models may further incorporate additional clinical factors that impact fertility outcomes, including sperm analysis, uterine conditions such as adenomyosis or endometriosis, as well as other patient-specific medical conditions, each of which may be assigned a relative weight within the analysis. In some embodiments, the AI models apply a weighted, multi-factor analytical framework informed by clinical research data to generate a balanced assessment of reproductive potential. The resulting analysis may be presented to and reviewed by a medical provider, who may approve, modify, or contextualize the predictive assessment during clinical consultation, or alternatively may be provided by the patient to a medical provider for independent evaluation.

In performing the analysis, the controller 200 correlates the biomarker data with additional diagnostic inputs, including follicle count information obtained through ultrasound examination, uterine anatomy findings derived from imaging studies, semen analysis results associated with a partner or donor, and available genetic testing results related to embryo development. These inputs are aggregated and evaluated collectively rather than in isolation, allowing the AI models to identify relationships among biomarkers, anatomical factors, and historical treatment outcomes that are not readily apparent through manual review.

Publicly available success rate data associated with a selected physician or clinic may also be incorporated. Diagnostic data may be manually entered by the user, uploaded by a medical provider, or received via application programming interfaces (APIs) connected to third-party laboratory or imaging systems.

During analysis, AI models executed by processor 220, optionally accelerated by GPU 250, identify patterns and correlations among biomarkers, demographic factors, and historical treatment outcomes. For example, AMH and FSH levels may be cross-referenced with age-based ovarian reserve models to detect diminished fertility scenarios, while uterine abnormalities may be flagged as potential implantation risk factors. The system may further identify missing, inconsistent, or conflicting data entries and generate prompts requesting clarification or additional testing.

In some embodiments, the controller 200 employs a multi-factor analytical framework to analyze patient-specific fertility data at step 304. Under this framework, heterogeneous data inputs, including demographic factors, diagnostic biomarkers, imaging findings, treatment history, and partner or donor information, are evaluated in parallel rather than sequentially by one or more trained artificial intelligence (AI) models. Each category of input contributes an independent analytical signal reflecting clinical relevance, risk, or feasibility, and the resulting signals are synthesized by the controller 200 into a unified analytical representation of the patient's reproductive profile. This parallel evaluation and synthesis enables the controller 200 to account for interactions among multiple fertility factors without requiring rigid rule-based processing or linear decision trees.

In further embodiments, certain clinical or diagnostic factors operate as eligibility-modifying or gating inputs within the multi-factor analytical framework. For example, uterine anatomy findings, ovarian reserve indicators, or recurrent treatment failure patterns may elevate, suppress, or condition the applicability of particular fertility treatment pathways without fully excluding other options. By incorporating such gating behavior into the analysis, the controller 200 dynamically adjusts the analytical weight or relevance of specific data inputs while preserving a comprehensive assessment across all available fertility pathways. This approach improves computational robustness and avoids oversimplified, binary determinations that can arise from manual or static evaluation techniques.

At step 306, the controller 200 generates a predictive fertility forecast assessment based on the analyzed patient-specific fertility data. In generating the predictive fertility forecast, the controller 200 utilizes one or more trained artificial intelligence (AI) models executed by processor 220 and optionally accelerated by GPU 250 to synthesize patient-specific fertility data and diagnostic information. In aspects, the predictive fertility forecast provides the patient with a clear, individualized assessment of the likelihood of IVF success and includes an estimated success rate expressed as a probability or percentage, a projected timeline reflecting anticipated treatment cycles and procedural milestones, and an estimated cost of IVF treatment that accounts for medication requirements, provider-specific pricing or, in some embodiments, a projected cost range based on geographic location and statistical pricing trends, and insurance coverage or lack thereof.

The predictive fertility forecast is generated by comparing patient data obtained at the outset of treatment to established benchmarks for success, including age-based fertility expectations, biomarker thresholds, and historical clinical outcomes associated with similar patient profiles. The predictive fertility forecast acts as the baseline profile for each individual patient. The forecast further incorporates the patient's unique financial position by factoring in the cost structure of the selected provider and applicable insurance benefits. In addition to assessing IVF success, the predictive fertility forecast includes alternative fertility treatment options for comparison, enabling evaluation of multiple potential pathways based on expected outcomes, timelines, and costs.

The predictive fertility forecast generated at step 306 is subject to evolution as additional indicators become available over the course of treatment. Such indicators may include outcomes associated with recruitment of healthy eggs and the number of eggs recruited, success of egg extraction procedures, fertilization of retrieved eggs to create embryos, sperm analysis results, changes in health and age (of both the patient and partner), whether the embryos reach blastocyst, and overall observed trends in embryo development during laboratory culture. As treatment progresses, each treatment cycle produces new clinical data, and the controller 200 compares data from each cycle to data from prior cycles and to stored benchmark outcomes. In some embodiments, the controller 200 further updates the predictive fertility forecast in response to changes in treatment protocols implemented by a medical provider, including modifications to medication regimens, dosing strategies, or procedural approaches. Such protocol changes may be incorporated as inputs to the AI models and reflected in updated success probabilities, timelines, and cost projections. In some embodiments, the controller 200 synchronizes forecast updates with cycle-specific treatment events and provider-specific treatment protocols, thereby maintaining a forecast that is continuously customized to the patient's ongoing treatment experience and the selected provider's approach. Through longitudinal comparison and trend analysis performed by the AI models, the predictive fertility forecast is continuously refined to evolve with the patient's individual treatment experience and to inform subsequent clinical and personal decision-making.

The predictive fertility forecast includes an estimated success rate of IVF expressed as a probability or percentage, a projected timeline reflecting anticipated treatment cycles and procedural milestones, and an estimated cost of IVF treatment that accounts for medication requirements, clinic pricing, insurance coverage, and procedural complexity. The forecast is generated using AI-based predictive models trained on population-level fertility outcome data stored within the system and correlated against the patient's individual profile, enabling an improved level of predictive accuracy relative to manual assessment techniques alone, while being configured for review, validation, and clinical interpretation by a medical provider. In some embodiments, the predictive fertility forecast may provide information to users to guide discussions with their doctors on how to approach IVF.

At step, 308 the controller 200 generates at least one alternative fertility treatment option based on the analyzed patient-specific fertility data. In performing this step, the controller 200 utilizes one or more trained artificial intelligence (AI) models executed by processor 220, and in some embodiments accelerated by GPU 250, to algorithmically evaluate whether alternative fertility pathways are viable or recommended in view of the patient's clinical profile, biomarker results, treatment history, and benchmark outcome data. The generation of alternative fertility treatment options includes determining whether surrogacy represents a clinically appropriate or advantageous pathway for the patient, either as a primary option or as a contingency to in-vitro fertilization. In making this determination, the controller 200 computes surrogacy-specific success likelihoods, projected timelines, and estimated costs by comparing the patient's analyzed fertility profile against population-level outcome data, surrogate dataset information, and stored treatment benchmarks. As part of step 308, the controller 200 prepares surrogacy as a selectable treatment pathway by associating the computed surrogacy parameters with the patient's profile, thereby enabling subsequent presentation and user selection. In this manner, surrogacy is generated as a computational output of the AI-driven analysis, rather than as a predefined or manually selected option, ensuring that alternative fertility treatment pathways are data-driven, individualized, and responsive to the patient's evolving clinical circumstances. In some embodiments, upon selection of surrogacy as a treatment pathway, the controller 200 further enables a guided surrogacy coordination workflow that supports user-directed management of the surrogacy process. As part of this workflow, the controller 200 may generate quantitative surrogate evaluation metrics based on medical (personal/family), demographic, and historical outcome data associated with potential surrogate candidates. These evaluation metrics may be presented to both the patient and a medical provider to support comparative assessment of surrogate candidates using objective, data-driven criteria, thereby supplementing clinical judgment and improving transparency, consistency, and decision-making in surrogate selection.

In some embodiments, generation of alternative fertility treatment options at step 308 is performed using the same multi-factor analytical framework applied during fertility data analysis, enabling comparative pathway evaluation within a unified computational process. The controller 200 evaluates in parallel the expected outcomes, timelines, and cost structures associated with in-vitro fertilization and one or more alternative fertility pathways, including surrogacy, based on the synthesized analytical representation of the patient's clinical profile. Rather than generating alternative options as isolated recommendations, the controller 200 produces a comparative assessment in which multiple pathways are scored and contextualized relative to one another using shared clinical, financial, and temporal factors.

In further embodiments, the controller 200 applies eligibility and feasibility conditions derived from patient-specific data to determine whether surrogacy or other alternative fertility pathways should be elevated, deprioritized, or surfaced as a recommended option. Such conditions may include medical compatibility thresholds, projected treatment burden, historical response trends, provider-specific constraints, and communication-derived indicators associated with the patient's evolving readiness to continue or pivot treatment pathways. In some embodiments, the controller 200 generates and updates a Pivot Readiness Index based on one or more of structured treatment data, free-form patient notes, direct communications, and community interactions, and uses the Pivot Readiness Index as an additional input when surfacing, prioritizing, or contextualizing alternative fertility treatment options. The resulting alternative fertility treatment options are synthesized into a unified output structure that preserves comparability across pathways while enabling individualized selection and discussion through the user interface. This data-driven comparative generation of fertility pathways improves the technological process of reproductive planning by enabling adaptive, context-aware pathway selection rather than static or user-driven navigation between treatment pathways performed without system-generated, data-driven prioritization.

At step 310, the controller 200 presents, via a user interface, the predictive fertility forecast generated at step 306 and at least one alternative fertility treatment option generated at step 308. The predictive fertility forecast includes a projected in-vitro fertilization (IVF) success rate expressed as a percentage likelihood, a timeline for expected treatment completion reflecting anticipated treatment cycles and procedural milestones, and an estimated cost of IVF treatment based on factors including medication requirements, insurance coverage or lack thereof, and clinic-specific billing information. The at least one alternative fertility treatment option may include surrogacy, egg donation or sperm donation, and/or adoption. In aspects, each alternative fertility treatment option may be presented with corresponding success likelihoods, projected timelines, and estimated costs as computed by the controller 200. In some embodiments, the user interface further presents educational content associated with each alternative fertility treatment option to support user understanding and facilitate informed discussion with a medical provider.

As the patient progresses through fertility treatment, the controller 200 updates the predictive fertility forecast generated at step 306 and the alternative fertility treatment option generated at step 308 based on additional clinical inputs received over time. Such inputs may include egg retrieval results, the number and quality of retrieved oocytes, fertilization outcomes, embryo development trends observed in a laboratory environment, and embryo transfer outcomes. The controller 200 incorporates these inputs to refine the information presented at step 310, enabling the displayed forecast and alternative fertility treatment options to evolve with the patient's individual treatment experience. In this manner, the system improves the technological process of IVF planning by providing adaptive, data-driven guidance through a networked computing platform, thereby enhancing accuracy in reproductive decision-making, reducing procedural delays, and minimizing unnecessary treatment cycles as compared to static or manually updated fertility evaluation techniques thereby enhancing accuracy in reproductive decision-making, reducing procedural delays, and minimizing unnecessary treatment cycles as compared to static or manually updated fertility evaluation techniques that are prone to data fragmentation, delayed updates, and inconsistent communication across patients and providers.

As the patient progresses through treatment, the predictive fertility forecast and the presented alternative fertility treatment options are continuously refined based on real-time inputs, including egg retrieval results, embryo quality assessments, and embryo transfer outcomes. In this manner, the system does not merely implement a static or mathematical model, but instead improves the technological process of IVF planning by providing real-time, data-driven, and adaptive guidance through a networked computing platform. This integrated approach enhances accuracy in reproductive decision-making, reduces procedural delays, and minimizes unnecessary cycle failures, thereby offering clear technical advantages over manual or static fertility evaluation techniques.

At step 312, the controller 200 facilitates, via the user interface, interactive communication between the user and at least one medical provider regarding the predictive fertility forecast generated at step 306 and the alternative fertility treatment options generated at step 308 and presented at step 310. The interactive communication is enabled through a shared framework implemented by the controller 200 and may include direct messaging between the user and the medical provider, exchange of medical documentation, presentation of annotated diagnostic results, or communications categorized by topic or level of urgency.

Through the interactive communication facilitated at step 312, the user and the medical provider may review, discuss, and coordinate decisions relating to the predictive fertility forecast and the alternative fertility treatment options, including clarification of clinical assumptions, discussion of risks and benefits, and coordination of next steps in treatment. In some embodiments, the system further receives updated treatment outcome data through the same communication framework, including egg retrieval results, embryo development metrics, or embryo transfer outcomes, and makes such data available for subsequent analytical processing by the controller 200. By integrating interactive communication with the computational outputs of the system, step 312 supports informed, coordinated reproductive decision-making and maintains alignment between automated analysis and clinical oversight throughout the fertility treatment process.

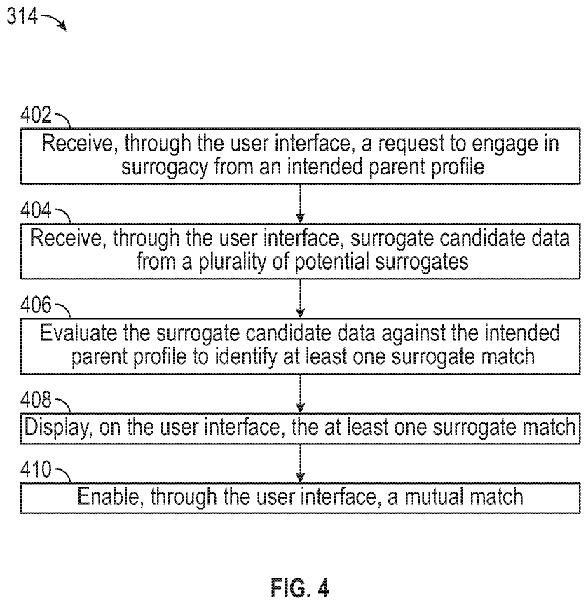

In some embodiments, following presentation of the alternative fertility treatment options at step 310 and interactive communication facilitated at step 312, the controller 200 initiates a surrogacy coordination and matching process at step 314 in response to the user selecting surrogacy as a fertility treatment pathway. The surrogacy coordination process is implemented by the controller 200 to expedite, simplify, and increase the accuracy and transparency of surrogate matching while reducing the cost, time, and administrative burden traditionally associated with third-party surrogacy agencies. An exemplary surrogacy coordination and matching workflow corresponding to step 314 is described in further detail with reference to FIG. 4.

Referring now to FIG. 4, an exemplary computer-implemented surrogacy coordination and matching method 314 is illustrated. The method is executed by controller 200 using at least one processor 220 in communication with memory 230 storing executable instructions. The surrogacy coordination and matching method is initiated in response to a user selecting surrogacy as a fertility treatment pathway, as described with respect to step 314 of FIG. 3. The disclosed method provides a technical solution to the technical problems of inefficient surrogate identification, fragmented communication, high agency costs, and lack of coordinated medical, legal, and psychological oversight in conventional surrogacy workflows.

Traditional surrogacy arrangements rely heavily on third-party agencies whose primary functions are surrogate identification and initial contract preparation, often followed by limited involvement throughout the remainder of the pregnancy and coordination processes that may be fragmented, inconsistently timed, or administratively burdensome. The present disclosure replaces or supplements these agency-centric processes with a computing platform that uses artificial intelligence, automation, structured data intake, and coordinated communication to algorithmically identify, evaluate, and manage surrogate matches while maintaining continuous connectivity among intended parents, surrogates, medical providers, and external service providers, including psychological, legal, and screening professionals. In some embodiments, such external services are performed by independent third-party providers, with the platform facilitating access, coordination, and transaction management without assuming responsibility for the underlying professional services. In further embodiments, the platform enables integrated financial workflows, including direct payment processing and fund management for fertility treatments and surrogacy-related services through partnerships with one or more financial institutions, thereby streamlining administrative processes and reducing transactional complexity and risk for users. In some embodiments, the controller 200 is configured to facilitate secure electronic payment transactions, including card-based payments, to clinics or other third-party providers participating in the reproductive pathway process.

At step 402, the controller 200 receives, through a user interface, a request to engage in surrogacy from an intended parent profile. The intended parent profile is generated through a user-facing application and includes structured data relating to medical eligibility, treatment history, geographic preferences, compensation constraints, insurance considerations, and personal or logistical preferences relevant to a surrogacy arrangement. In some embodiments, the intended parent profile further includes physician-or clinic-specific medical criteria that a surrogate must satisfy to be considered eligible for embryo transfer. The receipt of the surrogacy request initiates the surrogacy coordination workflow and signals the controller 200 to transition from fertility forecasting operations to surrogate identification and matching operations. The request may be submitted explicitly by user selection or implicitly based on AI-driven recommendations generated during fertility forecasting analysis. In some embodiments, the intended parent profile and corresponding surrogate profiles are generated using standardized questionnaire-based inputs and formatted into structured, templated profiles configured for use within the platform. Such profiles may be selectively distributed or promoted through external communication channels, including social media platforms, via platform-facilitated advertising or outreach mechanisms, thereby expanding visibility of intended parent and surrogate profiles while maintaining user-controlled privacy and consent settings. In further embodiments, the controller 200 may generate advertising-ready profile outputs derived from profile data, including persona-based or audience-targeted profile summaries that omit personally identifying information and are distributed only with user consent, thereby enabling platform-managed outreach beyond a members-only gallery while improving precision relative to generic mass-market surrogate recruitment approaches.

At step 404, the controller 200 receives, through the user interface, surrogate candidate data from a plurality of potential surrogates. Each surrogate candidate generates a surrogate profile using a standardized data input framework provided by the platform. The surrogate candidate data includes biographical information, medical history, obstetric history, psychological screening status, geographic location, availability, insurance information, and compensation expectations. In some embodiments, the surrogate candidate data further includes results of preliminary medical screenings, psychological evaluations, reason(s) for wanting to be a surrogate, and legal eligibility checks conducted by qualified professionals. These preliminary evaluations are used to establish an initial level of candidate qualification prior to further clinical, legal, and procedural vetting required for formal surrogate matching. The platform may provide templates and guided prompts to ensure that surrogate data is entered in a consistent, structured manner, enabling accurate “apples-to-apples” comparisons across surrogate candidates. All surrogate candidate data is stored in database 210 and made available for algorithmic evaluation. In some embodiments, the platform provides predefined input templates and guided prompts for both intended parent profiles and surrogate profiles, enabling standardized data capture that facilitates machine-learning model training and consistent, normalized comparison across users.

The evaluation process considers medical compatibility criteria defined by fertility clinics or obstetric providers, including uterine health, obstetric history, and risk factors; psychological compatibility criteria derived from screening outcomes and preferences; legal eligibility requirements based on jurisdiction; financial parameters including fair-market compensation caps; and logistical considerations such as geographic proximity and availability. In some embodiments clinic-specific criteria are received via user input or uploaded documentation and incorporated into the evaluation process to enable customized, provider-aligned matching.

At step 406, the controller 200 evaluates the surrogate candidate data against the intended parent profile to identify at least one surrogate match. This evaluation is performed using one or more trained artificial intelligence (AI) models executed by processor 220 and, in some embodiments, accelerated by GPU 250. The AI models mine, aggregate, and analyze the surrogate candidate data and intended parent data to identify predictive factors associated with successful surrogate pregnancies and compatible surrogate, intended parent relationships.

The AI models are trained using historical surrogate pregnancy outcomes, clinical benchmarks, and platform-specific success metrics to prioritize surrogate candidates with a higher likelihood of successful pregnancy and sustained cooperation throughout the surrogacy journey. In some embodiments, such compensation parameters are constrained within predefined fair-market value ranges enforced by the controller 200 and agreed upon during user onboarding, thereby discouraging bidding dynamics and promoting equitable access to surrogate candidates

At step 408, the controller 200 displays, on the user interface, at least one surrogate match identified at step 406. The displayed surrogate match includes a structured presentation of surrogate profile information, compatibility indicators, medical alignment with provider criteria, estimated timelines, and anticipated cost parameters. The presentation is designed to provide intended parents with sufficient information to evaluate suitability while maintaining privacy and compliance with applicable regulations. In some embodiments, the user interface further provides links to vetted psychological, legal, and medical support resources localized to the surrogate and intended parent, enabling informed review of next-step requirements.

In some embodiments, the user interface may further provide access to on-demand informational content and platform-based community resources, including discussion interfaces and user-to-user communication features, enabling users to access educational materials and shared experiences in a structured and accessible format.

In further embodiments, the controller 200 may generate and display forecast-derived compatibility or process-readiness information for review by the surrogate, thereby enabling the surrogate to assess the intended parent profile, expected treatment pathway, and associated coordination factors using the same or a related data-driven framework applied to intended parent decision support.

At step 410, the controller 200 enables, through the user interface, the intended parent and surrogate are presented with a mutual match, sharing the profile of the intended parent with the surrogate and the surrogate profile with the intended parent for review and consideration. A mutual match is established only upon affirmative approval by both the intended parent and the surrogate. Upon mutual approval, the platform initiates coordinated communication workflows and unlocks access to shared medical records, messaging channels, and scheduling tools. The coordinated communication workflows are in accordance with HIPAA compliance requirements and mutual consent. Following the establishment of the mutual match, the controller 200 facilitates ongoing coordination among the surrogate, intended parents, fertility clinic, obstetric provider, laboratories, psychological and legal professionals, and genetic counselors. The forecast may support the evaluation of the surrogate, and provide quantitative data that is additive to the clinic's criteria and industry standards. In some embodiments, the controller 200 further provides a guided process management interface including automated checklists, milestone tracking, and reminder generation associated with medical, legal, insurance, and screening requirements of the surrogacy process. The controller 200 may synchronize completion status of such process steps across the intended parent and surrogate profiles, thereby ensuring coordinated progression through required stages. Payments associated with the surrogacy arrangement may be routed through the platform, and direct payment financial management tools may be enabled. In embodiments, connectivity to escrow partners that offer attorney insight and strict compliance may be included. Throughout the surrogacy process, the platform maintains shared access to medical records and pregnancy-related data, enabling continuous monitoring, communication, and documentation. In some embodiments, the controller 200 coordinates timing and release of such payments based on predefined milestones, confirmations, or mutual approvals associated with the surrogacy process. In further embodiments, the controller 200 may facilitate secure electronic payments to clinics, screening providers, legal professionals, or other third parties through one or more partnered financial institutions or banking services.

By implementing the surrogacy coordination and matching method as a data-driven, AI-enabled workflow integrated with fertility forecasting and provider communication systems, the disclosed invention improves the technological process of surrogacy planning and execution. The system improves the relationship between the intended parent and surrogate, contributes to reduced stress on the pregnant surrogate and intended parents, eliminates reliance on manual agency processes, lowers administrative overhead, improves matching accuracy, enhances transparency, and enables continuous, coordinated oversight across medical, legal, psychological, and financial dimensions of surrogate pregnancies.

In some embodiments, the controller 200 enforces matching constraints, including limiting the number of simultaneous matches presented to intended parents, imposing time-based decision windows, and preventing duplicate presentation of the same surrogate candidate to multiple intended parents concurrently. These constraints promote fairness, prevent bidding dynamics, improve overall matching efficiency, and reduce the likelihood of incompatible or unstable matches.

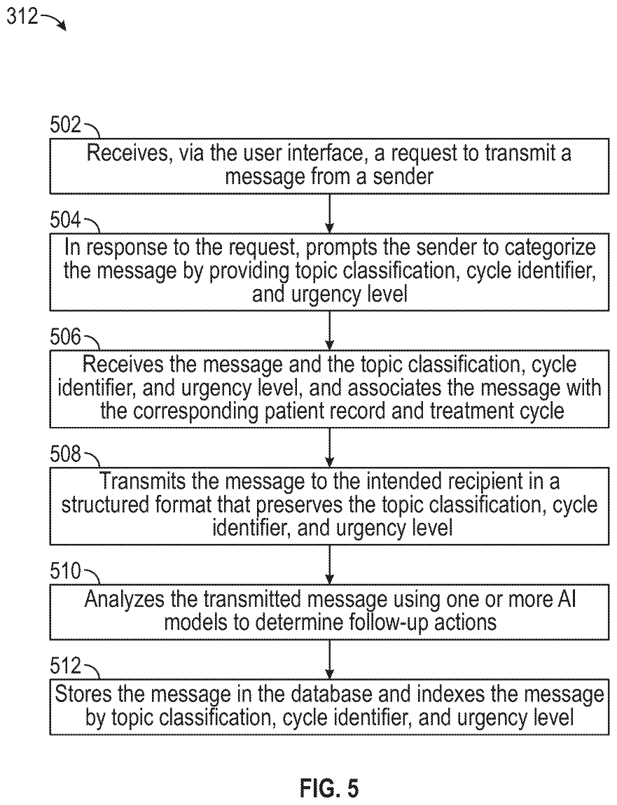

Referring now to FIG. 5, an exemplary computer-implemented communication method 312 is illustrated that expands upon and implements the interactive communication step described at step 312 of FIG. 3. The method 312 is executed by controller 200, which includes at least one processor 220 and memory 230 storing instructions that, when executed, cause the controller 200 to perform the operations described herein. The disclosed communication framework provides a technical solution to the problem of fragmented, unstructured, and time-sensitive fertility-related communications by programmatically enforcing standardized message categorization, cycle-aware data association, and automated follow-up messages and appointments using automation and artificial intelligence (AI). By linking communications directly to treatment cycles, clinical context, and urgency parameters, the system improves the technological process by which fertility-related information is transmitted, reviewed, and acted upon between users and medical providers, thereby enhancing coordination, reducing missed or delayed responses, and improving continuity of care throughout the fertility treatment process.

At step 502, the controller 200 receives, via a user interface, a request to transmit a message from a sender. The sender may include a patient, an intended parent, a surrogate, or a medical provider. The request may be initiated through a mobile application, web-based interface, or other user device 140 in communication with the controller 200 over network 130.

At step 504, in response to the request to transmit the message, the controller 200 prompts the sender to categorize the message prior to transmission. The required categorization includes selection of a topical classification, identification of an associated treatment cycle and cycle date, and designation of a level of message urgency. In some embodiments, the urgency level includes predefined urgency classifications corresponding to response expectations, including a low urgency classification indicating a response timeframe of approximately forty-eight to seventy-two hours, a medium urgency classification indicating a response timeframe of approximately twenty-four hours, and a high urgency classification indicating a same-day or immediate response timeframe. In further embodiments, the user interface provides a dedicated note-taking and checklist environment associated with the patient profile, enabling the patient, surrogate, and intended parent, to record free-form thoughts, questions, observations, or treatment-related reflections independent of structured message transmission. Such notes may be associated with a treatment cycle, stored within database 210, and maintained alongside structured communications and treatment data.

In some embodiments, the topical classification selected at step 504 corresponds to one or more predefined topic categories associated with fertility treatment workflows. Such topic categories may include, without limitation, cycle-specific discussions integrated with all relevant clinical information for the identified cycle, diagnostic results associated with a treatment cycle, treatment protocols linked to corresponding medication lists, medication lists linked to treatment protocols, calendar-based topics including appointment dates and times, and consult-related topics including recommendations provided by physicians or nurses during consultations. In aspects, consult-related topics further include question-and-answer content generated during consultations and transcribed or summarized using AI-based note generation. In further embodiments, the controller 200 may analyze the stored free-form notes using one or more AI models to identify patterns, sentiment, or emerging concerns associated with the patient's treatment experience. In some embodiments, such analysis contributes to generation or updating of a Pivot Readiness Index that reflects evolving patient readiness, treatment burden, or interest in alternative pathways. Based on such analysis, the system may prompt the user to organize selected notes into a structured communication, including generating a draft message for transmission to a medical provider or suggesting scheduling of a consultation. In some embodiments, communications received from medical providers may be automatically associated with and incorporated into the patient's stored notes, thereby maintaining a centralized and continuously updated record of treatment-related information. The user interface may further enable search and retrieval of both structured messages and stored notes, allowing the patient to access prior communications, instructions, or observations across treatment cycles.

At step 506, the controller 200 receives the message content together with the selected topic classification, cycle identifier, cycle date, and urgency level, and associates the message with the corresponding patient record and treatment cycle stored in database 210. The association enables the message to be contextually linked with other clinical, scheduling, and treatment-related data for the identified cycle, thereby preserving continuity of care and reducing ambiguity in subsequent review.

At step 508, the controller 200 transmits the message to the intended recipient in a structured format that preserves the selected topic classification, cycle identifier, cycle date, and urgency level. By transmitting the message together with structured metadata, the system ensures that recipients receive communications within the appropriate clinical and temporal context, improving response accuracy and reducing delays caused by missing or unclear information.

At step 510, the controller 200 analyzes the transmitted message using one or more artificial intelligence (AI) models executed by processor 220 and, in some embodiments, accelerated by GPU 250, to determine follow-up actions associated with the message. In aspects, the AI models interpret the message content and associated metadata to identify conditions requiring follow-up alerts, including unanswered questions, unacknowledged messages, pending appointment reminders, or outstanding diagnostic or laboratory results. The controller 200 may generate alerts or reminders based on this analysis and associate such alerts with the corresponding patient record and treatment cycle. Following transmission and analysis, the controller 200 stores the message in database 210 and indexes the message by topic classification, cycle identifier, cycle date, and urgency level. This indexing enables retrieval of messages through cycle-specific views, topic-based organization, or search-based access within the user interface. In this manner, the platform maintains a consolidated, cycle-aware communication record that allows patients and providers to access relevant information directly, such as viewing medication-related communications, reviewing prior consult notes, or searching historical messages associated with a particular treatment cycle.

In some embodiments, the structured communication framework further integrates patient guidance resources accessible through the user interface. Such resources may include instructional materials relating to medication administration, definitions of prescribed medications, descriptions of medication side effects, community support content and coping mechanisms. In aspects, medical providers may upload additional patient-specific guidance materials tailored to an individual treatment protocol, which may be referenced within communications or used by AI systems to support consistent responses to predictable patient questions.

In some embodiments, the communication framework may further include live communication features and support resources, including live chat sessions with social workers, patient support forums, and live question-and-answer interactions facilitated by an AI-driven virtual nurse. In aspects, responses generated by the AI-driven virtual nurse are shaped using stored patient guidance materials and clinical protocols, and may be transmitted to a corresponding medical provider for review, oversight, or follow-up. The platform may further provide community-based features, including message boards, on-demand content, and a community dashboard customized to a user profile based on one or more of treatment needs, age, and location, thereby improving relevance and reducing information overload. In some embodiments, community access may include localization features enabling users to participate in local, regional, national, or international communities based on zip code or other geographic parameters, and subscription-based access tiers may enable expanded community capabilities. Differentiated user interfaces may be provided for patients, intended parents, and surrogates to accommodate role-specific communication needs.

By enforcing structured message categorization, cycle-aware data association, AI-driven follow-up determination, and indexed storage, the communication method illustrated in FIG. 5 improves the technological process of clinical communication in fertility treatment. The disclosed framework reduces missed communications, improves responsiveness to time-sensitive events, and enhances coordination among patients, providers, and ancillary participants, thereby providing clear technical advantages over unstructured messaging systems.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different example embodiments provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A system for reproductive pathway forecasting and coordination, comprising:
a processor; and
a memory, including instructions stored thereon, which when executed by the processor cause the system to:
receive, from a user device, patient-specific fertility data from a patient, including known indicators and biomarkers of fertility;
analyze the patient-specific fertility data;
generate a predictive fertility forecast assessment based on the analyzed patient-specific fertility data, the predictive fertility forecast including an estimated success rate of IVF, a cost associated with IVF treatment, and a projected timeline of IVF treatment; and
present, on the user interface, the predictive fertility forecast and at least one alternative fertility treatment option, wherein the at least one alternative fertility treatment option includes surrogacy.

2. The system of claim 1, wherein the known indicators and biomarkers of fertility include at least one of anti-mullerian hormone (AMH) levels, follicle-stimulating hormone (FSH) levels, age, weight, height, advanced genetic embryo testing, and semen analysis.

3. The system of claim 1, wherein the instructions, when executed by the processor, further cause the system to:
generate an updated predictive fertility forecast based on at least one of recruitment of healthy eggs from the patient, fertilization of the healthy eggs to create an embryo, and success of embryo development in a lab.

4. The system of claim 1, wherein the instructions, when executed by the processor, further cause the system to:
facilitate, by the user interface, interactive communication between the user and at least one medical provider regarding the predictive fertility forecast and the at least one alternative fertility treatment option.

5. The system of claim 1, wherein the instructions, when executed by the processor, further cause the system to:
receive, through the user interface, a request to engage in surrogacy from an intended parent profile;
receive, through the user interface, surrogate candidate data from a plurality of potential surrogates;
evaluate the surrogate candidate data against the intended parent profile to identify at least one surrogate match; and
display, on the user interface, the at least one surrogate match; and
enable, through the user interface, a mutual match.

6. The system of claim 5, wherein a mutual match includes:
the intended parent profile approving and the at least one surrogate match; and
the at least one surrogate match approving the intended parent profile.

7. The system of claim 1, wherein the interactive communication between the user and the at least one medical provider is a shared framework between the user and the at least one medical provider.

8. The system of claim 7, wherein the shared framework includes a direct message between a sender and a receiver, and the sender is at least one of the users and the at least one medical provider.

9. The system of claim 8, wherein the sender may categorize the direct message by topic and a level of urgency for the receiver to respond.

10. A computer-implemented method for providing an in-vitro fertilization (IVF) forecast, the method comprising:
receiving, from a user device, patient-specific fertility data, including known indicators and biomarkers of fertility;
analyzing the patient-specific fertility data;
generating a predictive fertility forecast assessment based on the analyzed patient-specific fertility data, the predictive fertility forecast including an estimated success rate of IVF, a cost associated with IVF treatment, and a projected timeline of IVF treatment; and
presenting, on the user interface, the predictive fertility forecast and at least one alternative fertility treatment option, wherein the at least one alternative fertility treatment option includes surrogacy.

11. The method of claim 10, wherein the known indicators and biomarkers of fertility include at least one of anti-mullerian hormone (AMH) levels, follicle-stimulating hormone (FSH) levels, age, weight, height, advanced genetic embryo testing, and semen analysis.

12. The method of claim 10, further comprising:
generating an updated predictive fertility forecast based on at least one of recruitment of healthy eggs from the patient, fertilization of the healthy eggs to create an embryo, and success of embryo development in a lab.

13. The method of claim 10, further comprising:
facilitating, by the user interface, interactive communication between the user and at least one medical provider regarding the predictive fertility forecast and the at least one alternative fertility treatment option.

14. The method of claim 10, further comprising:
receiving, through the user interface, a request to engage in surrogacy from an intended parent profile;
receiving, through the user interface, surrogate candidate data from a plurality of potential surrogates;
evaluating the surrogate candidate data against the intended parent profile to identify at least one surrogate match; and
displaying, on the user interface, the at least one surrogate match; and
enabling, through the user interface, a mutual match.

15. The method of claim 14, wherein a mutual match includes:
the intended parent profile approving and the at least one surrogate match; and
the at least one surrogate match approving the intended parent profile.

16. The method of claim 10, wherein the interactive communication between the user and the at least one medical provider is a shared framework between the user and the at least one medical provider.

17. The method of claim 16, wherein the shared framework includes a direct message between a sender and a receiver, and the sender is at least one of the users and the at least one medical provider.

18. The method of claim 17, wherein the sender may categorize the direct message by topic and a level of urgency for the receiver to respond.
